(19) United States
(12) Patent Application Publication
(10) Pub. No.: US 2007/0140924 A1
(43) Pub. Date: Jun. 21, 2007

(54) Maintenance Free Automatic Odor Control Device and System

(76) Inventor: Micael John Hill, Cedar Falls, IA (US)

Correspondence Address: PATWRITE LLC, 408 W. MAIN ST., MARSHALLTOWN, IA, 50158-5759, US

(21) Appl. No.: 11/612,406

(22) Filed: Dec. 18, 2006

Related U.S. Application Data
(60) Provisional application No. 60/750,474, filed on Dec. 16, 2005.

(51) Int. Cl. A61L 9/12

(52) U.S. Cl. 422/124

(57) ABSTRACT

A maintenance free automatic odor control system comprises a flow through fabric sheet having an attachment end to attach to a forced air heating and cooling register. The attachment end may be a magnetic strip of it may be half of a Velcro® pull with the other half mounted on the register. As air is forced through the register, the scent is dispersed throughout the room. Additionally, the fabric is porous and also filters particulates and other impurities from being released into the room by capturing them in the weave of the fabric. The sheets are disposable and are simply replaced and discarded when no longer effective. Because the filter fabric is chosen to be aesthetically pleasing, otherwise unattractive registers may be hidden.

### RELATED APPLICATIONS

This application claims priority and herein incorporates by reference U.S. provisional patent application 60/750,474, filed Dec. 16, 2005.

### BACKGROUND OF THE INVENTION

The present invention relates to the field of home, business and

transportation ventilation systems, and more particularly to a confined or interior area air freshener, odor reducer to be used in conjunction with vent or air registers.

Pre-scented fragrance impregnated fibrous disposable sheets and more particularly, methods, which pertains to HAVC venting register systems. Whereas the scented disposable sheet is positioned and attached on exteriors of vent registers. Every since humans began living in enclosed areas, the problem of odors was encountered, although our ancestors seemed to have more tolerance for odors than is acceptable to our modern sensibilities.

It is known to use scent dispersal methods and systems in homes, offices, public establishments and vehicles. Such devices and methods often included stationary table scenting devices and plug in configurations. Most of these represent inconvenient solutions at best or noticeable devices set up on furniture, counters, shelves or wall outlets. Other methods include application of spray aerosols under pressure, pumped or inert gases are common, along with drops, heated scented oil, themes, jells, and other liquid scents. Stationary jell methods either evaporate over a period of time or are heated to displace the scenting fragrance. Liquid types often are sprayed or misted in the air for circulation of the inert fragrance to control odors or foul olfactory smells.

Often these scent dispersal methods need a form of outside energy to initiate the method of dispersal, such as electricity or human interaction such as pumping, spraying in the intended enclosed area. Although these methods work, they have many have limitations which prevent a user from fully enjoying a maintenance free odor masking and removal solution.

Another method of choice involves heating a potpourri which evaporates to release aromatic pleasing scents in the intended enclosed area. Many of these systems work strictly on either heated elements or evaporation methods and as such are not circulated actively under fan convection.

There is a need for a method of scent dispersal incorporating a convenient, simple maintenance free automatic odor control system and method which acts as a filtering agent and scenting device and overcomes the limitations in existing art.

### SUMMARY OF THE INVENTION

A maintenance free automatic odor control device and system comprises a flow through fabric sheet having an attachment end to attach to a forced air heating and cooling register. The attachment end may be a magnetic strip of it may be half of a Velcro® pull with the other half mounted on the register. As air is forced through the register, the scent is dispersed throughout the room. Additionally, the fabric is porous and also filters particulates and other impurities from being released into the room by capturing them in the weave of the fabric. The sheets are disposable and are simply replaced and discarded when no longer effective. Because the filter fabric is chosen to be aesthetically pleasing, otherwise unattractive registers may be hidden.

Foregoing disadvantages inherent in known types of scenting devices now present, the present invention overcomes the adversities of prior art and provides a very simple and cost effective method of dispersing fragrance through utilization of existing interior HAVC resources commonly available and used in interior or confined areas. In the aforementioned, this system provides a very convenient and accessible method, which utilizes pre-existing HAVC vents & registers within enclosed areas. The present instant invention provides register vent method scenting system and air filtering method wherein the pre-scented disposable sheets our affixed firmly in a temporary manner to the exterior vents or registers and acts in either passive or active modes.

The pre-scented fragrance imbedded fibrous disposable sheets are comprised of a fibrous embodiment impregnated with a variety of pleasant fragrances. Each fibrous sheet is lightly layered to form its body and is semi porous in that air can pass through in a slightly obstructed flow. Each sheet has a front or back to it's basic rectangular embodiment design depending on the intended affixation type embodiment. The back or rear sheets linier body length enables an attachment method to affix to built-in HAVC supplying systems in interior structures such as a home, office, store, restaurant or transportation vehicle. The scented disposable sheet impregnated with a pleasant fragrance is attached to the HAVC venting system by an affixation method stable to affix the sheet for intended use and allow the sheet to be removed and/or replaced with a new, over time, scenting sheet. The scenting method will disperse the scent fragrance through the interior structure in a passive mode such exampled in prior art in stationary scented jells or scented card embodiments. When the HVAC system becomes active automatically or manually through its HAVC fan system, such like heating, air conditioning or general air fan circulation. The circulating, moving air in the intended enclosure, home, office, rv, or restaurant changes the pre-scented fragrance imbedded fibrous disposable sheets from a passive scenting method to an active circulatory and semi gathering particulate filtering device. The impregnated fragrance sheet will further disperse evaporation properties due to the airflow through the sheet and embodiment. With this active HAVC method the scented sheets also form a filtering agent for air borne particulates. The filtering sheet by method and placement will gather a variety of particulates through the fibrous semi-porous scenting sheet.

Other features and advantages of the instant invention will become apparent from the following description of the invention, which refers to the accompanying drawings.

### BRIEF DESCRIPTION OF THE DRAWINGS

FIG. 1 is a top view of a disposable scent dispersal and filter sheet according to an embodiment of the present invention.

FIG. 2 is a perspective view of the disposable scenting dispersal and filter sheet affixed to a HAVC vent system during operation.

FIG. 3 is an enlarged view of a cross-section of a portion of a disposable scent dispersal and filter sheet according to an embodiment of the present invention

FIG. 4 is a perspective view of a disposable scent dispersal and filter sheet according to an embodiment of the present invention.

FIG. 5 is a side view of the disposable scent dispersal and filter sheet according to an embodiment of the present invention.

FIG. 6 is a perspective view of a disposable scent dispersal and filter sheet according to an embodiment of the present invention.

FIG. 7 is a perspective view of a disposable scent dispersal and filter sheet according to an embodiment of the present invention.

FIG. 8 is a perspective view of a disposable scent dispersal and filter sheet according to an embodiment of the present invention.

FIG. 9 is a top view of a disposable scent dispersal and filter sheet according to an embodiment of the present invention.

FIG. 10. Is a side view of a disposable scent dispersal and filter sheet according to an embodiment of the present invention.

### DETAILED DESCRIPTION OF THE INVENTION

Reference is now made to the drawings in which reference numerals refer to like elements.

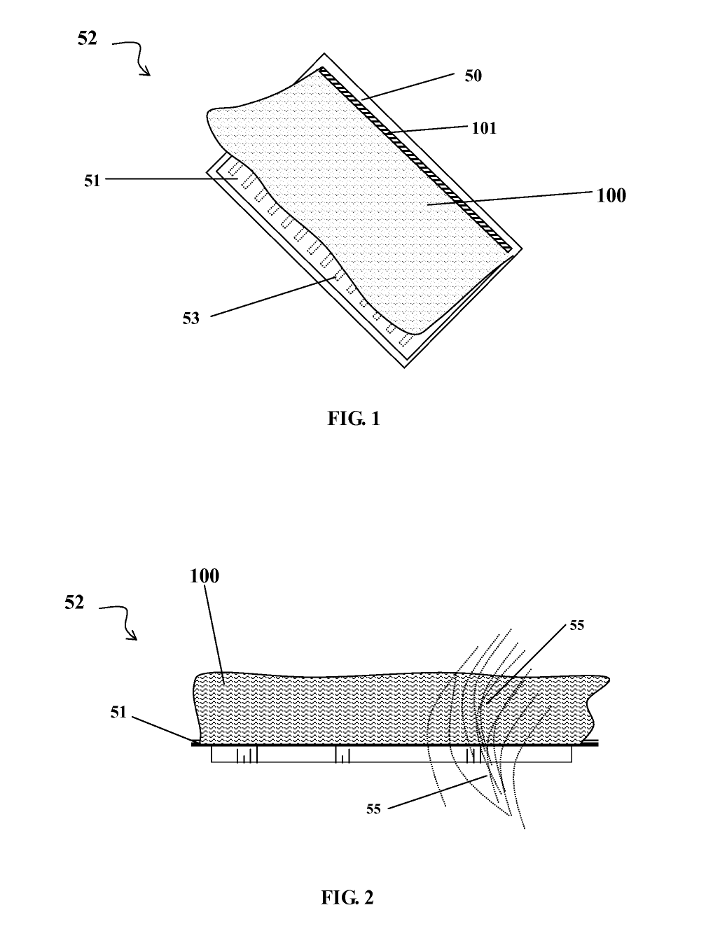

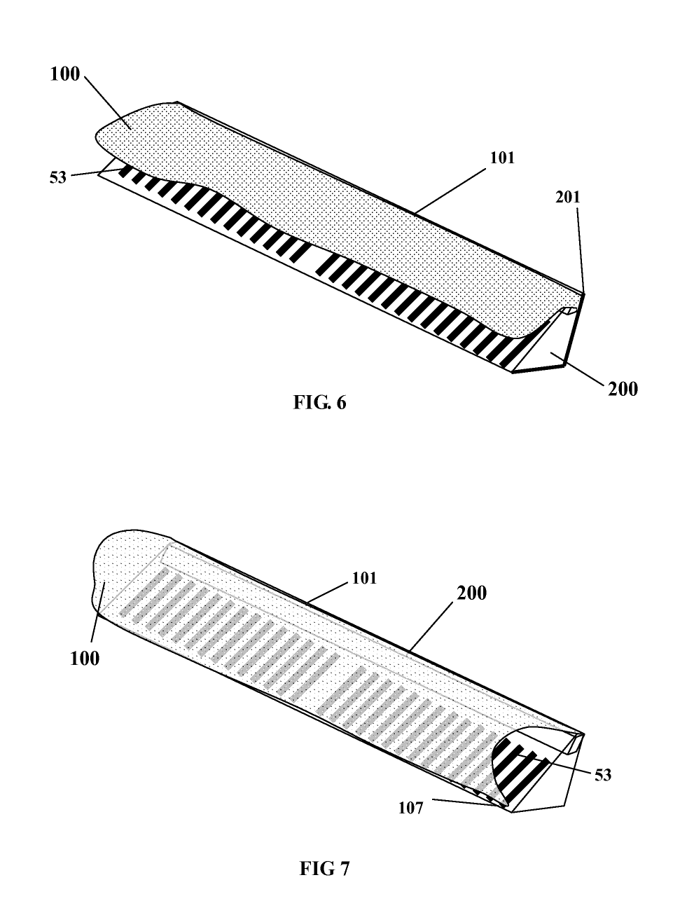

Referring to FIGS. 1 through 5, a disposable scent dispersal and filter sheet 100 is shown attached to a common HAVC vent 51. Vent 51 is normally mounted to the floor mounted, as well as the wall and ceiling in some applications. This embodiment comes in a variety of styles however are mostly standard for homes, offices and restaurants. Differences are more discerned in Automobile dash vent sizes and RV HAVC systems for heating. FIGS. 6 and 7 are common home and office vents. This style comes in different. This design style is for floor edges and walls, “where they meet”. Both basic styles may direct air from an inlet source beneath the vent and direct it into the room or space through a plurality of slots 53. Other options allow a user to shut or reduce the airflow with an adjustment mechanism within the vent apparatus. Typical styles include a top area or side area whereas a portion of vent 51 is available for attachment methods of the scenting device.

A disposable scent dispersal and filter sheet is installed in a flattened position for attachment to the floor/wall/ceiling register 51. Disposable scent dispersal and filter sheet 100 attached to a rear top fastening area 50. Means of attachment include tape, glue, clips, magnets, slides, attachments, sticky strips, hook and loop fastener (like Velcro®) or other acceptable adhesive material 101 as is known in the art.

Referring now to FIGS. 6 and 7, a disposable scent dispersal and filter sheet 100 is composed of a fibrous compound and is attached singularly or dual method by attaching to a top or bottom portion 201, 107 respectively or by both in a dual attachment method.

Now referring to FIGS. 1 and 2, airflow 55 is shown directed through slots 53 and impacting disposable scent dispersal and filter sheet 100. Disposable scent dispersal and filter sheet 100 is uplifted by convection currents through register 52.

Referring now to FIGS. 2, 8 and 10, a disposable scent dispersal and filter sheet 100 is shown having a fabric mesh structure which allows air 55 to pass through but captures particulates within the weave. The movement of air 55 also disperses a scent which is embedded within disposable scent dispersal and filter sheet 100.

FIG. 9 shows an embodiment of the present invention where disposable scent dispersal and filter sheet 100 is attached both on a top edge 107 and a bottom edge 120. In this embodiment, as the air passes through plurality of slots 53, disposable scent dispersal and filter sheet 100 inflates to direct airflow through fabric weave thereby trapping particulates and releasing a scent. When disposable scent dispersal and filter sheet 100 is no longer effective, it is discarded and a new sheet is installed.

Referring to FIGS. 1, 7 & 9 one can see two very common HAVC styled venting apparatus, 52 and 200. FIG. 1 This inherent design is normally for floor mounted HAVC and also walls & ceilings. This embodiment comes in a variety of styles, however mostly standard for homes, offices and restaurants. Differences are more discerned in Automobile dash vent sizes and RV HAVC systems for heating and possible cooling. FIGS. 7 and 9 are common home and office types venting apparatus's. This style comes in different sizes and embodiments however commonly standard for the aforementioned. This design style is for floor edges and walls, “where they meet”. Both basic styles, 52, 200 may direct air from an inlet source beneath the vent and direct it into the room or space through slots as shown in 53. Other options are shutting or reducing the flow with an adjustment mechanism within the vent apparatus. Typical styles include a top area or side area whereas a portion of the embodiment is available for attachment methods of the scenting device, shown in 50,51,201 and 202.

Referring now to FIGS. 2,3,4,5,8,10,11,12 and 13 the substrate-scenting sheet is laid in a flattened position for attachment to the floor/wall/ceiling register, 52 and 200. Scented fibrous sheet is then attached by the preferred embodiment to the rear top fastening area 50, 201. Means of attachment include tape, glue, clips, magnets, slides, attachments, sticky strips, velcro, adhesive materials as shown in 101, 201, 202, and 109. The attachment means may be either both or separately on the scenting sheet 100 or the registers 52 and 200. Different and assorted attachment methods means are well known in prior art.

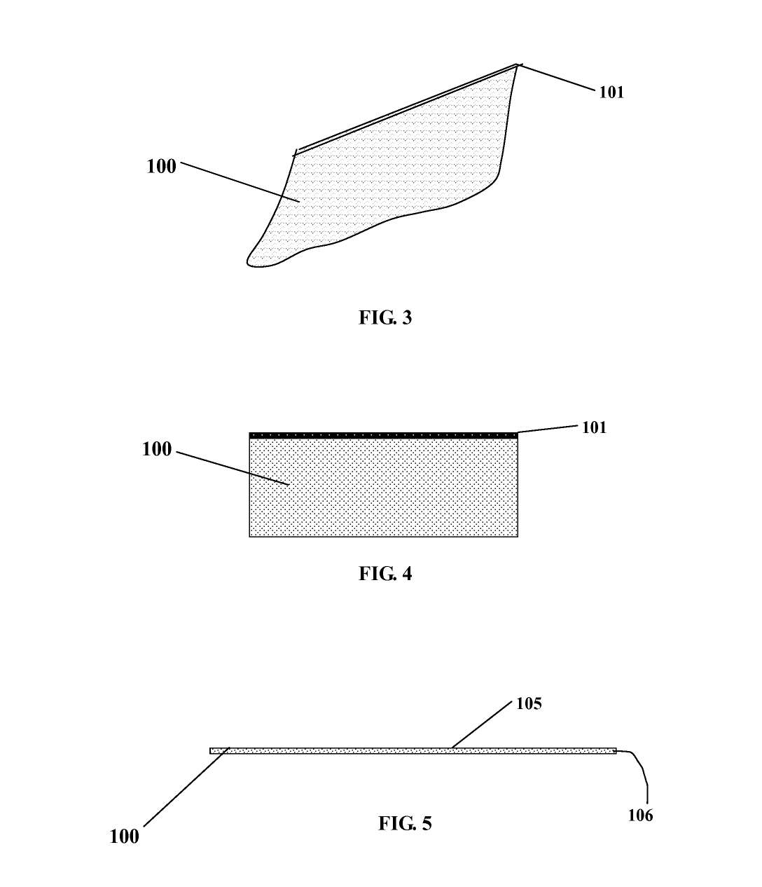

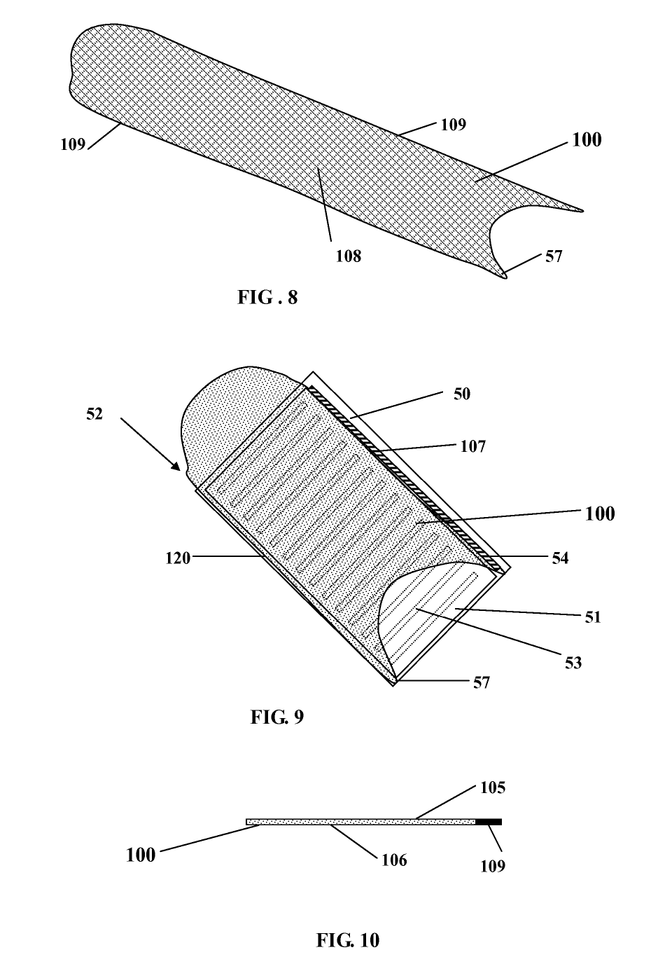

Referring now to FIGS. 2,3,4,5,8,10,11,12 and 13 the scented sheet 100 is composed of a fibrous compound and is attached singularly or dual method by aforementioned attachment to 51 and 200 styled venting apparatus. FIG. 2 shows scented sheet 100 uplifted by convection currents through the register 52 continuing through the air slots 53 in the described active mode of air scenting and filtering. Singular attachment by 101 is temporally affixed as also shown is FIG.3 and 8.

Referring now to FIG. 3 showing a side view illustration of 52, a common floor HAVC register whereas, air is blowing up from the inlet area, reacting with 100 for which becomes active in further disbursement of fragrance as shown and 100 becomes a filtering body. This is also shown in FIGS. 8, 10 and 12. In the active mode, air particulates our captured along 100 and within its porous fibrous embodiment.

Referring to FIG. 10 whereas the scenting sheet is attached to 101 and 107 in this attachment method the scenting sheet 100 is attached in two separate means on 52 and/or 200. In this embodiment 100 becomes a scenting sheet in conjunction with a filtering agent. 101 and 107 further define attachment area points.

Scenting and filtering sheet 100 can be made of a variety of existing materials, from woven fiber compounds to non-woven even including types of foam compounds. 100 may also be precut or sized where the user may cut the sheet to fit their particular application means.

Illustrations and different colors can also be incorporated into the sheets to assist in complementing the visual embodiment. This could include stripping the sheets, different designs, perforations, colors, textures and corner designs. Scenting sheet 100 may also be impregnated with a large variety of chemical compounds in reducing particulates in confined areas. This would assist in reductions, odors, molds, pollens, viruses, bacterial agents, dust, smoke, pet dander, allergens and other known interior hazards and distractions.

Many common existing scenting systems fail to utilize the resources within the confined area, namely the HAVC system vents, 52 200 that in either the active or passive modes has by incorporation by most common construction, exists within confined areas. It's readily availability and disposal may be further utilized. Pre-Scented disposable sheet system 100, allows user an easy and simple approach to fragrance dispensing and particulate reduction within, for example their homes. User removes the prepackaged scented sheet and applies in a temporary attachment method the sheet atop the register vent, FIG. 2 and 8. As aforementioned they may also attach to the bottom attachment area, FIGS. 10 and 12, creating internal hollow area for particulate entrapment. In either method the scenting sheet will disperse fragrance with the confined area. In the active mode, airflow 55, this will further increase fragrance dispersal. When the scenting sheet 100, has lost it's inherent scenting ability the user simply removes the sheet from the vent 52, 200 and replaces with a new one.

Many options may be employed for the eye candy effect these sheets 100 may have from colors, perforations, stripping and textures. In different embodiments of 100 one could have endless styles and designs to assist in correlating the interior decor and further allowing covering unsightly registers in style and design.

The present invention may be made of foam and similar materials known to those in the art and may be attached to the register vent system and chemical compounds may be sprayed on the sheet to assist in a longer life or additional olfactory pleasing fragrances and odor reducers. The present invention uses fragrance formulations which include evaporation modifiers, enhancers or retardants to control the evaporation process. The filter sheets may be impregnated entirely or partially of, microencapsulated fragrance oils.

The filter sheets may be woven or non-woven, porous or synthetic carrier material that holds and allows evaporative release of fragrance. The sheets may be cut in strips, die-cut designs, perforations or other decorative designs. Other means of attachment include secure but temporary fasteners like clips, attachments, tapes, glues, magnetic means, sticky strips, Velcro, adhesive materials and others known in existing art. The filter sheet may include an encapsulated jell disposed within the top and bottom of the scenting sheet.

When the present invention is secured at both sides, the filter sheet inflates in the air flow and defines a semi enclosed area for capture of particulates. Airflow movement is then directed horizontally. Other embodiments of the filter sheet include a non-woven fabric substrate formed from substantially continuous filaments having a denier within the range of 1.5 to 35 denier per filament. The filaments include a matrix of filaments formed of polyester homopolymer and binder filaments formed of a polyester copolymer, and a multiplicity of bonds throughout the fabric at locations where the binder filaments contact other filaments. Additionally, the filter sheet may incorporate nanoparticles known to be capable of destroying bacteria, fungi, viruses, or toxins. The nanoparticles are combined with the scenting qualities and active filtering. The filter sheets may be impregnated with a variety of olfactory compounds, pleasing scents, fragrances, antibacterial, deodorizing and perfumes as is known in the art. The filter sheet may also utilize static electricity to collect dust and other compounds as is known in the art. Additionally, chemical compounds may be utilized to assist collecting smoke, dust, allergens, molds, funguses, pet dander, pollens and other microscopic particulates common in enclosed or confined areas.

A scented air register system comprised of scented sheets applied exteriorly to interiorly contained/enclosed HAVC registers and vent systems. Each vent sheet is pre-scented and a disposable fibrous embodiment, fragrance or perfume impregnated. Attached exteriorly on registers and or vents of a central heat, air conditioner system, main vent systems or enclosed transportation modes. Method of scent is dispersed by applying the pre-scented aromatic disposable sheet to the vent or register by temporary affixation method whereas the scented sheet is either passive or active.

In a passive mode the scented sheet will disperse the fragrance by olfactory sensing evaporation, such as like paper or jell scenting methods. The active mode will action when the ventilations system activates the fan and air flow through and around the porous nature of the scented sheet will deodorize, freshen, scent, mask odors and slightly filter the circulating ambient air within the intended interior space.

Although the instant invention has been described in relation to particular embodiments thereof, many other variations and modifications and other uses will become apparent to those skilled in the art.

What is claimed is:

1. A disposable scent dispersal and filter sheet comprising:
a filter sheet having an attachment portion along a side;
at least one attachment means disposed on said attachment portion whereby said sheet is attachable to at least one of a top and bottom of a forced air register; and
said filter sheet being infused with a scent whereby said scent is dispersed by airflow therein.

2. A disposable scent dispersal and filter sheet according to claim 1 wherein said at least one attachment means is a magnet.

3. A disposable scent dispersal and filter sheet according to claim 1 wherein said at least one attachment means is a hook and loop fastener.

4. A disposable scented sheet, which is according to claim 1 wherein said bodies' inert compound composition is supple and flexible.

5. A disposable scented sheet, which is according to claim 1 wherein said size is die-cut shape and/or die-cut perorations.

6. A disposable scented sheet, which is according to claim 1 wherein said embodiment has an affixation method to the rear lateral.

7. A disposable scented sheet which is according to claim 1 wherein said embodiment has a affixation method to the front lateral and combined together as stated in claim 4 for the rear affixation.

8. A disposable scented sheet, which is according to claim 1 wherein, has a graphic imprint of colors and/or designs and/or motifs.

9. A disposable scented sheet, which is according to claim 1 wherein, said is semi-porous in compensation to allow capture of particulates. The sheet being made of a material which will absorb fragrances and which is flexible, embodiment of the scenting sheet allowing the fragrance to emit by evaporation.

10. A disposable scented sheet, which is according to claim 1 wherein said embodiment inherently may be impregnated with olfactory fragrances.

11. A disposable scented sheet which is according to claim 1 wherein said embodiment is fibrous in the micro capsulated fragrance oil.

12. A disposable scented sheet, which is according to claim 1 wherein said embodiment is impregnated with a microbe-sterilizing agent.

13. A disposable scented sheet which is according to claim 1 wherein said embodiment in a active mode is a air filtering method.

14. A disposable scented sheet, which is according to claim 1 wherein said embodiment may be manufactured and packaged in precut, precut perforated rolls.

15. A disposable scented sheet which is according to claim 1 wherein said embodiment may be manufactured as precut rectangular sheets intended for common sized air register and vent systems.

16. A disposable scented sheet which is according to claim 1 wherein said embodiment displays the option of 2 methods to affix directly to a air register system HAVC venting embodiment.

17. A disposable scented sheet, which is according to claim 1 wherein said embodiment is further, includes four sides manufactured with curved corners as to the style desire.

18. A disposable scented sheet which is according to claim 1 wherein said embodiment.

19. A disposable scented sheet which is according to claim 1 wherein said embodiment is aligned atop the register by means of affixation.

20. A disposable scented sheet, which is according to claim 17 wherein scenting sheet is substantially aligned with said register venting air flow apertures.
